(19) United States
(12) Patent Application Publication
(10) Pub. No.: US 2022/0203809 A1
(43) Pub. Date: Jun. 30, 2022

(54) VEHICULAR DISPLAY CONTROL DEVICE

(71) Applicant: DENSO CORPORATION, Kariya-city (JP)

(72) Inventors: Ifushi SHIMONOMOTO, Kariya-city (JP); Ryohei YOKOTA, Kariya-city (JP); Yuji OTA, Kariya-city (JP)

(21) Appl. No.: 17/583,049

(22) Filed: Jan. 24, 2022

Related U.S. Application Data
(63) Continuation of application No. PCT/JP2020/029082, filed on Jul. 29, 2020.

(30) Foreign Application Priority Data
Jul. 29, 2019 (JP) 2019-138973
Jul. 10, 2020 (JP) 2020-119135

(51) Int. Cl. B60J 3/04; B60K 35/00

(52) U.S. Cl. CPC B60J 3/04; B60K 35/00; B60K 2370/175; B60Y 2200/11; B60K 2370/785; B60K 2370/27; B60K 2370/149; B60K 2370/152

(57) ABSTRACT

The vehicular display control device is mounted on an autonomous driving vehicle provided with a windshield display. The windshield display is configured to change a transmittance of external light. The vehicular display control device includes a transmittance control unit configured to reduce the transmittance in order to promote a sleep of at least one passenger.

### CROSS REFERENCE TO RELATED APPLICATION

The present application is a continuation application of International Patent Application No. PCT/JP2020/029082 filed on Jul. 29, 2020, which designated the U.S. and claims the benefit of priority from Japanese Patent Applications No. 2019-138973 filed on Jul. 29, 2019 and No. 2020-119135 filed on Jul. 10, 2020. The entire disclosures of all of the above applications are incorporated herein by reference.

### TECHNICAL FIELD

The present disclosure relates to a display control device mounted on a vehicle.

### BACKGROUND

In recent years, with the development of automatic driving technology of vehicles, a technology for improving the comfort in the vehicle compartment has been proposed. In a conceivable technique, various information presentation devices such as a digital mirror and a head-up display are proposed. In addition, a windshield display that displays information on the entire surface of the windshield overlapped on the background has also been proposed.

### SUMMARY

According to an example embodiment, a vehicular display control device is mounted on an autonomous driving vehicle provided with a windshield display. The windshield display is configured to change a transmittance of external light. The vehicular display control device includes a transmittance control unit configured to reduce the transmittance in order to promote a sleep of at least one passenger.

### BRIEF DESCRIPTION OF THE DRAWINGS

The above and other objects, features and advantages of the present disclosure will become more apparent from the following detailed description made with reference to the accompanying drawings. In the drawings:

FIG. 1 is a diagram showing a compartment of an autonomous driving vehicle according to the first embodiment;

FIG. 2 is a block diagram showing a configuration of a vehicle compartment environment establishment system according to the first embodiment;

FIG. 3 is a flowchart showing a vehicle compartment environment control process according to the first embodiment;

FIG. 4 is a flowchart showing a determination process of a target passenger according to the first embodiment;

FIG. 5 is a flowchart showing a sleep onset process according to the first embodiment;

FIG. 6 is a flowchart showing a sleep process according to the first embodiment;

FIG. 7 is a flowchart showing an awakening process according to the first embodiment;

FIG. 8 is a flowchart showing another example of the awakening process according to the first embodiment;

FIG. 9 is a flowchart showing an emergency awakening process according to the first embodiment;

FIG. 10 is a diagram showing an example of a transmittance pattern controlled for each region of WSD in order to promote sleep onset according to the first embodiment;

FIG. 11 is a diagram showing a vehicle compartment of an autonomous driving vehicle at the time of awakening processing according to the second embodiment;

FIG. 12 is a flowchart showing an awakening process according to the second embodiment; and

FIG. 13 is a flowchart showing another example of the awakening process according to the second embodiment.

### DETAILED DESCRIPTION

It is thought that fully autonomous driving will be possible in the future. In a vehicle in which fully autonomous driving is performed, it may be desirable to develop a vehicle compartment environment so that passengers including the driver can effectively use the vehicle compartment. As a result of detailed examination by the present inventors, it has been found that the above-mentioned technology is insufficient to establish a vehicle compartment environment.

In view of the above points, it may be desirable that a vehicle compartment environment suitable for passengers can be established by using a windshield display. The vehicular display control device of one aspect of the present embodiments is mounted on an autonomous vehicle provided with a windshield display. The windshield display is configured so that the transmittance of external light can be changeable. The vehicular display control device includes a transmittance control unit configured to reduce the transmittance in order to promote the sleep of at least one passenger.

According to the vehicle display control device of one aspect of the present embodiments, the transmittance of the windshield display is reduced in order to promote the sleep of the passenger. By reducing the transmittance of the windshield display, the incident of outside light into the vehicle compartment is suppressed. Therefore, the passenger can quickly fall asleep and take a sufficient nap in the passenger compartment. That is, by controlling the windshield display as an environment establishment device in the vehicle compartment, it is possible to establish a vehicle compartment environment suitable for the passengers.

Hereinafter, exemplary embodiments for implementing the present disclosure will be described with reference to the drawings.

### First Embodiment

<1. Configuration of Vehicle Compartment Environment Establishment System>

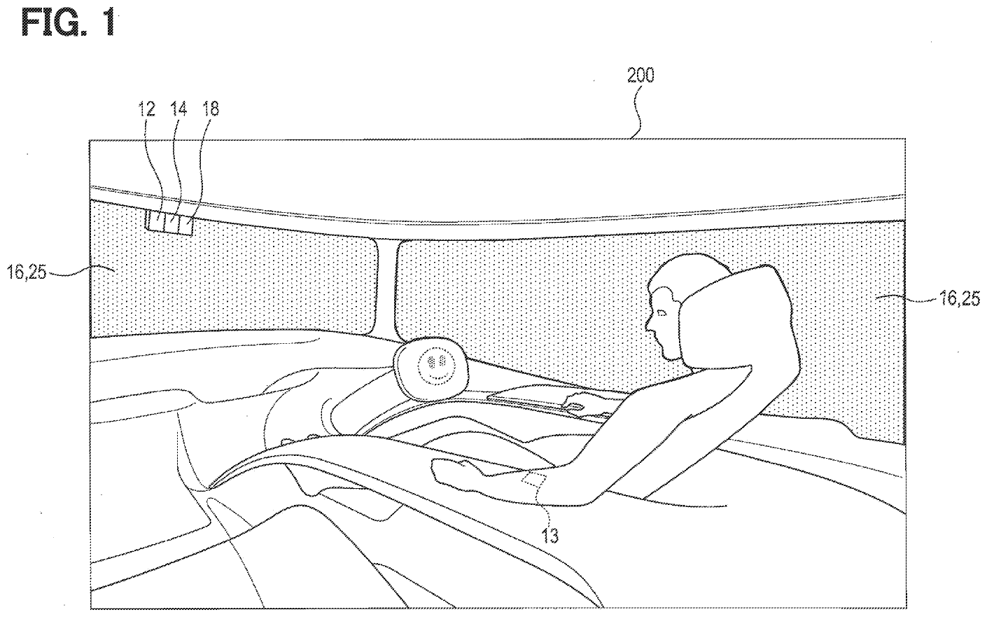

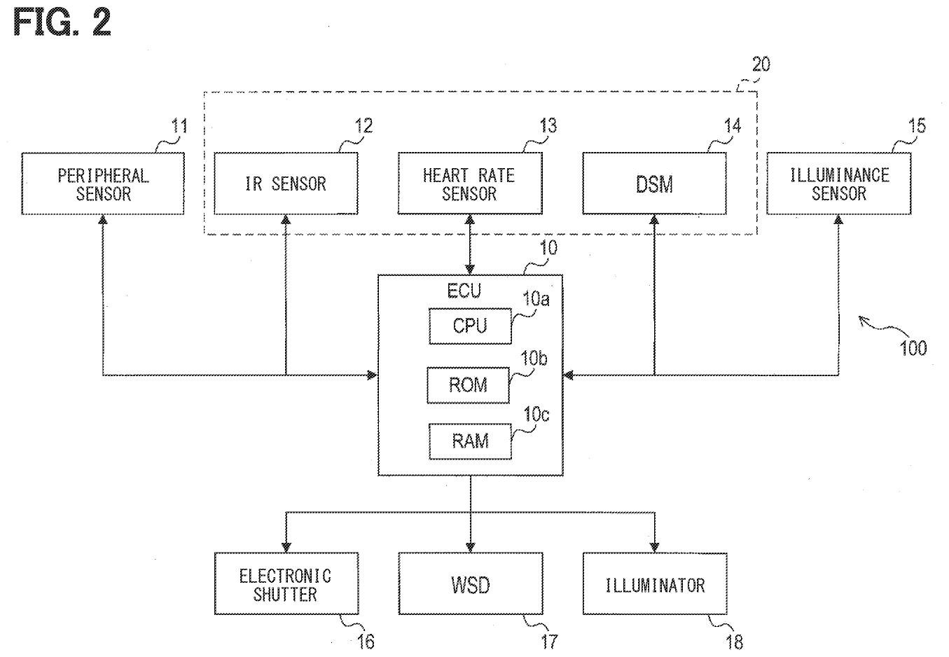

First, the configuration of the vehicle compartment environment establishment system 100 according to the present embodiment will be described with reference to FIGS. 1 and 2. The vehicle compartment environment establishment system 100 is mounted on the autonomous driving vehicle 200. In the present embodiment, the autonomous driving vehicle 200 is capable of autonomous driving at level 4 or higher according to the Society of Automotive Engineers standard.

The vehicle compartment environment establishment system 100 includes an electronic control unit (hereinafter, ECU) 10, a peripheral monitoring sensor 11, a biological sensor 20, an illuminance sensor 15, an electronic shutter 16, a windshield display (hereinafter, WSD) 17, and an illuminator 18.

The peripheral monitoring sensor 11 is a sound wave sensor (that is, sonar), a laser radar, a millimeter wave radar, an image sensor, or the like. The peripheral monitoring sensor 11 is mounted, for example, in the center of the front bumper of the autonomous driving vehicle 200, or on the left side or the right side of the front bumper. The peripheral monitoring sensor 11 detects obstacles such as other vehicles existing around the autonomous driving vehicle 200, and transmits the detection information to the ECU 10.

The biosensor 20 is a sensor that detects the biometric information of the passenger, and includes an IR sensor 12, a heartbeat sensor 13, and a driver status monitor (hereinafter, DSM) 14. In the present embodiment, the biometric information includes the body temperature, facial expression, and heartbeat of the passenger.

The IR sensor 12 corresponds to a radiation thermometer that absorbs infrared rays emitted by an passenger and measures the body temperature of the passenger. The IR sensor 12 is mounted in the center and above the vehicle width direction, for example, inside the front windshield 25. When a plurality of passengers get on the autonomous driving vehicle 200, the IR sensor 12 measures the body temperature of each passenger. Then, the IR sensor 12 measures the body temperature of the passenger at a predetermined cycle, and transmits the measured temperature information to the ECU 10.

The DSM 14 corresponds to a camera that captures a facial image of an passenger including a driver. The DSM 14 is mounted in the center and above the vehicle width direction, for example, inside the front windshield 25. When a plurality of passengers are on the autonomous driving vehicle 200, the DSM 14 captures a face image including the faces of the plurality of passengers. Alternatively, the DSM 14 may be equipped with a camera provided for each seat. The DSM 14 captures a face image of the passengers at a predetermined cycle, and transmits the captured face image of the passengers to the ECU 10.

The heart rate sensor 13 is a sensor that detects the pulse of the passenger. The heart rate sensor 13 is mounted at a position where the passenger comes into contact with each seat of the autonomous driving vehicle 200. Specifically, the heart rate sensor 13 is mounted on the armrest that the passenger's arm contacts and the seat surface that the passenger's femoral portion contacts, and detects the pulse of the passenger's arm and thigh. The heart rate sensor 13 detects the passenger's pulse information (that is, heart rate information) at a predetermined cycle, and transmits the detected pulse information to the ECU 10.

The biosensor 20 may not include the heart rate sensor 13. In this case, the movement of the blood vessels of the face may be detected from the facial image of the passenger taken by the DSM14, and the pulse may be calculated.

The illuminance sensor 15 is a sensor that detects the illuminance of light. The illuminance sensor 15 is mounted in a place that receives a large amount of external light, such as the inside of the front windshield 25 or the back side of the rear view mirror, and detects the illuminance of the external light. The illuminance sensor 15 detects the illuminance at a predetermined cycle and transmits the detected irradiation amount information to the ECU 10. In this embodiment, the illuminance information corresponds to the external environmental information.

The electronic shutter 16 is made of a plurality of film members, and is attached to the entire windshields 25 on the front side, the left side, the right side, and the rear side in a grid pattern. Each film member has, for example, a square shape. The transmittance of the electronic shutter 16 can be changed stepwise by applying a voltage. The electronic shutter 16 may be built in the windshield 25. That is, the windshield 25 may be configured so that the transmittance can be changed stepwise by applying a voltage.

The WSD 17 projects display light indicating various information onto the windshields 25 on the front side, the left side, the right side, and the rear side of the autonomous driving vehicle 200. As a result, the display light reflected by the electronic shutter 16 and the external light (that is, sunlight) transmitted through the electronic shutter 16 are directed to the eyes of the passenger. As a result, the passenger recognizes the display light as a virtual image displayed and overlaid on the external landscape. The windshield 25 and the electronic shutter 16 function as projection target members on which display light is projected. Various types of information include road information, safety information, navigation information, vehicle information, entertainment information such as movies, and the like.

The WSD 17 is configured so that the transmittance of external light can be changed by providing the windshield 25 with an electronic shutter 16. The visibility of the external landscape and the visibility of the display light by the passenger vary depending on the transmittance of the external light.

When the transmittance of the electronic shutter 16 is increased, the amount of external light transmitted through the windshield 25 increases, and the amount of display light reflected by the windshield 25 decreases. Therefore, the visibility of the external landscape is high, and the visibility of the display light is low. When the transmittance of the electronic shutter 16 is set to 100%, the passenger cannot see the display light.

When the transmittance of the electronic shutter 16 is decreased, the amount of external light transmitted through the windshield 25 decreases, and the amount of display light reflected by the windshield 25 increases. Therefore, the visibility of the external landscape is low, and the visibility of the display light is high. When the transmittance of the electronic shutter 16 is set to the lowest value (specifically, a value close to 0%), the external light transmitted through the windshield 25 is almost eliminated. As a result, the inside of the vehicle compartment becomes dark, and the passenger visually recognizes the display light projected on the windshield 25. That is, by making the transmittance of the electronic shutter 16 close to 0, the inside of the vehicle compartment can be darkened and the windshield 25 can be used as a screen.

Further, the electronic shutter 16 can adjust the transmittance for each film member. Therefore, it is not necessary to adjust the entire windshield 25 to the same transmittance, and the windshield 25 can be divided into a plurality of regions and the transmittance can be adjusted for each of the plurality of regions. Therefore, it is possible to reduce the transmittance in the region where the external light reaches the eyes of some of the passengers among the plurality of passengers, and not to change the transmittance in the other regions.

The illuminator 18 is mounted in the center and above the vehicle width direction, for example, inside the front windshield 25. The illuminator 18 includes a plurality of light emitting members such as LEDs, and when the selected light emitting member among the plurality of light emitting members is turned on, the face of the selected passenger among the plurality of passengers is irradiated with light.

The ECU 10 includes a CPU 10a, a ROM 10b, a RAM 10c, an I/O, and the like, and the CPU 10a executes various programs stored in the ROM 10b to provide the functions of a transmittance control unit, a biometric information acquisition unit, a drowsiness determination unit, and an awakening determination unit, an external information acquisition unit, a lighting control unit, and an image display unit.

The ECU 10 determines the state of each passenger using the acquired biometric information. Then, the ECU 10 controls the transmittance of the WSD 17 (that is, the transmittance of the electronic shutter 16) according to the determined state of each passenger to establish a vehicle compartment environment suitable for the state of each passenger. Here, three modes are defined as the passenger's state: a sleep onset mode in which the passenger falls asleep, a sleep mode in which the passenger is in a sleep state, and an awakening mode in which the passenger is awakened. Further, the awakening mode includes a normal awakening mode and an emergency awakening mode for awakening in an emergency. Further, the ECU 10 has a function of Artificial Intelligence, learns the usage pattern of the passenger compartment for each passenger, and constructs a more suitable passenger compartment environment for each passenger. In this embodiment, the ECU 10 corresponds to a vehicle display control device.

<2. Vehicle Compartment Environment Control Processing>

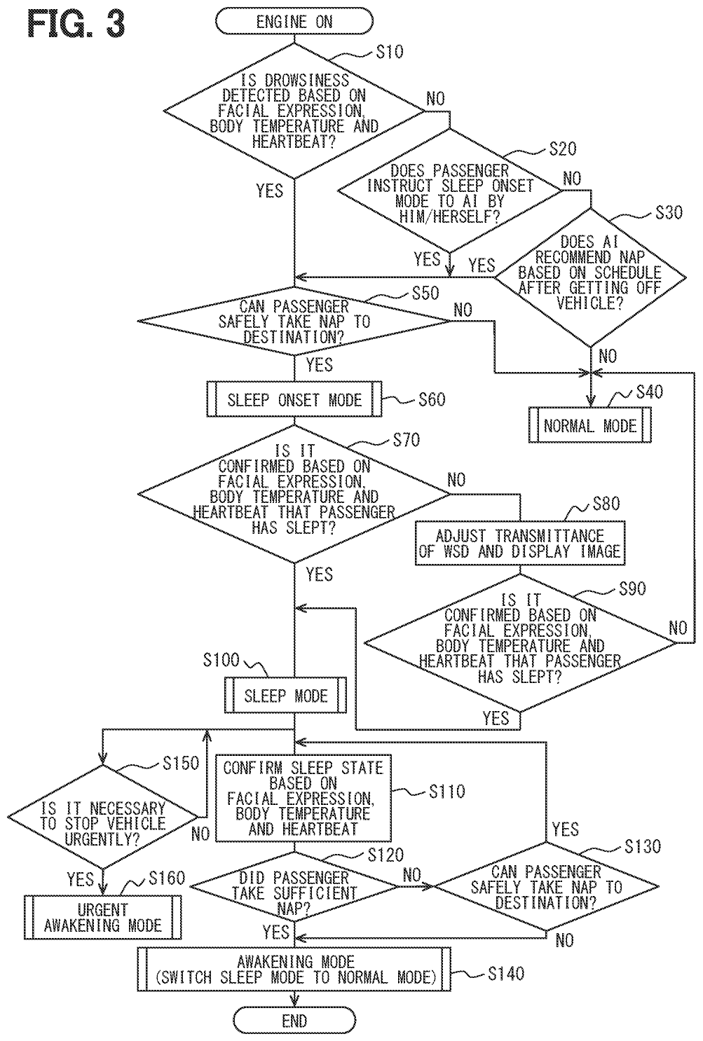

Next, the vehicle compartment environment control process executed by the ECU 10 will be described with reference to the flowcharts of FIGS. 3 to 9.

First, in S10, the facial expression, body temperature, and heartbeat of each passenger are acquired, and it is determined whether or not the drowsiness of each passenger is detected based on the acquired facial expression, body temperature, and heartbeat of each passenger. When it is determined in S10 that the drowsiness of each passenger has not been detected, the process proceeds to the process of S20, and when it is determined that the drowsiness of each passenger has been detected, the process proceeds to the process of S50.

In S20, it is determined whether or not any of the passengers has instructed the ECU 10 to fall asleep mode to put the passenger to sleep. The sleep onset mode may be instructed by any means such as switch input, touch panel input, and voice input. When it is determined in S20 that the sleep onset mode has been instructed, the process proceeds to S50, and when it is determined that the sleep onset mode has not been instructed, the process proceeds to S30.

In S30, it is determined whether or not to recommend a nap to each passenger based on the schedule after each passenger gets off the vehicle. The schedule after each passenger gets off the vehicle may be input by each passenger, or the ECU 10 may be linked with the smartphone of each passenger so that the ECU 10 may automatically acquire the schedule from the smartphone. Further, the schedule after getting off of each passenger may be acquired from the result that the AI function of the ECU 10 has learned from the past behavior of each passenger.

The ECU 10 recommends a nap when each passenger performs a high-load work such as a long time business meeting after getting off the vehicle, and does not recommend a nap when resting at home. When it is determined in S30 that nap is recommended, the process proceeds to S50, and when it is determined that nap is not recommended, the process proceeds to S40.

In S40, the normal mode is turned on. Specifically, the transmittance of the WSD is set to the standard transmittance so that the passenger can recognize the display light displayed and superimposed on the external landscape.

On the other hand, in S50, it is determined whether or not it is possible to safely take a nap to the set destination. Specifically, road information, weather information, and the like are acquired by wireless communication with the information center, and it is determined whether or not the vehicle can travel to the destination by level 4 automatic driving. In S50, when it is determined that the nap cannot be safely performed, the process proceeds to S40, and when it is determined that the nap can be safely performed, the process proceeds to S60.

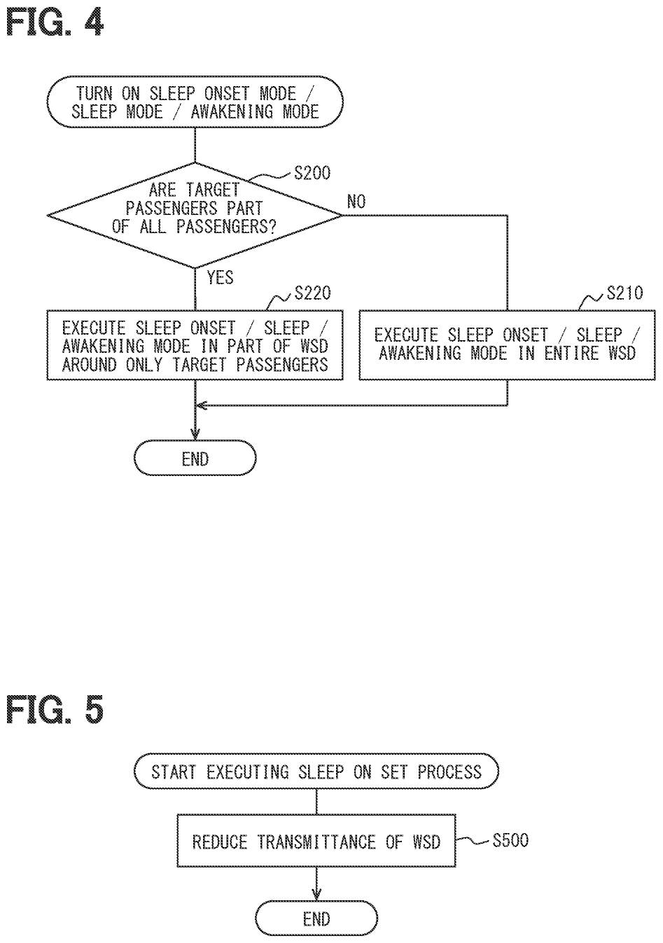

In S60, the sleep onset mode is turned on and the flowchart shown in FIG. 4 is executed. First, in S200, it is determined whether or not the passenger who is to fall asleep is a part of the passengers. In S200, when it is determined that all the passengers are to fall asleep, the process proceeds to S210, and when it is determined that some passengers are to fall asleep, the process proceeds to S220.

In S210, the sleep onset process shown in the flowchart of FIG. 5 is executed in the entire area of the WSD 17, that is, the entire windshield 25. Specifically, in S500, the transmittance is lowered in the entire region of WSD17. As a result, less outside light enters the vehicle compartment and the vehicle compartment becomes dark. In the sleep onset mode, the transmittance of the WSD 17 is lowered to reduce the external light that reaches the passenger's eyes and encourage the passenger to fall asleep.

On the other hand, in S220, the sleep onset process shown in the flowchart of FIG. 5 is executed in a part of the WSD17. Specifically, in S500, the transmittance of a part of the WSD17 is lowered. A part of the WSD17 is a part of the WSD17 corresponding to a sleeping passenger, and is a part where the outside light reaches the passenger's eyes. This reduces the external light that reaches the eyes of the passengers who is to fall asleep, but does not reduce the external light that reaches the eyes of other passengers.

Returning to the flowchart of FIG. 3, in S70, the facial expression, body temperature, and heartbeat of each passenger are acquired, and it is determined whether it can be confirmed that the passenger who has entered the sleep onset mode has slept based on the acquired facial expression, body temperature, and heartbeat of each passenger. When it is determined in S70 that the sleep of the passenger has not been confirmed, the process proceeds to S80, and when it is determined that the sleep of the passenger has been confirmed, the process proceeds to S100.

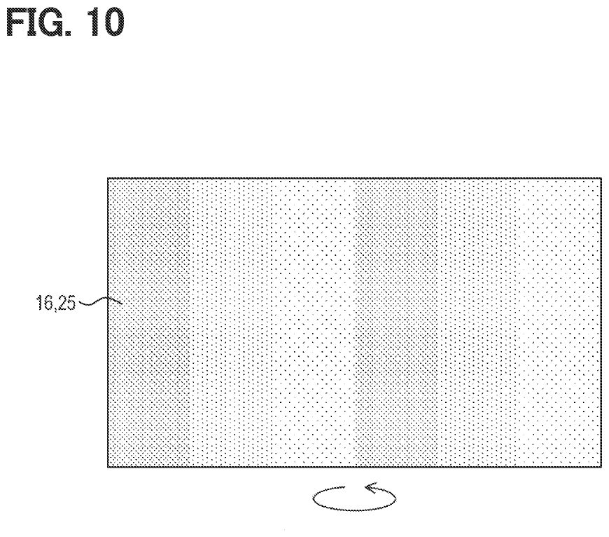

In S80, the transmittance of WSD17 is adjusted in order to encourage the passenger who has entered the sleep onset mode but has not slept. Specifically, a plurality of regions are set in the WSD 17 (that is, the windshield 25), and the transmittance is changed with time for each of the set plurality of regions. For example, as shown in FIG. 10, a plurality of regions divided in the vehicle width direction are set in the WSD 17, and the transmittances of the adjacent regions are set to different transmittances. As shown in FIG. 10, at one point in time, the transmittances of the six regions are set to low, medium, high, low, medium, and high, and at the next time point, the transmittances of the six regions are set to medium, high, low, medium, high, and low. In this way, the transmittance of each region is adjusted so that regions having different transmittances appear to move in the vehicle width direction. Alternatively, in order to encourage the passenger who has entered the sleep onset mode to sleep, an image that induces drowsiness is displayed on the WSD17 that has become a screen with the transmittance of the WSD17 set to the minimum value.

Subsequently, in S90, as in S70, it is determined whether or not it is determined whether it is conformed that the passenger has slept. When it is determined in S90 that the sleep of the passenger has not been confirmed, it gives up to make the passenger to sleep, and the process proceeds to S40, and it turns on the normal mode. On the other hand, when it is determined in S90 that the sleep of the passenger has been confirmed, the process proceeds to S100.

In S100, the sleep mode is turned on and the flowchart shown in FIG. 4 is executed. First, in S200, it is determined whether or not the sleeping passengers are a part of the passengers. In S200, when it is determined that all the passengers are sleeping, the process proceeds to S210, and when it is determined that some passengers are sleeping, the process proceeds to S220.

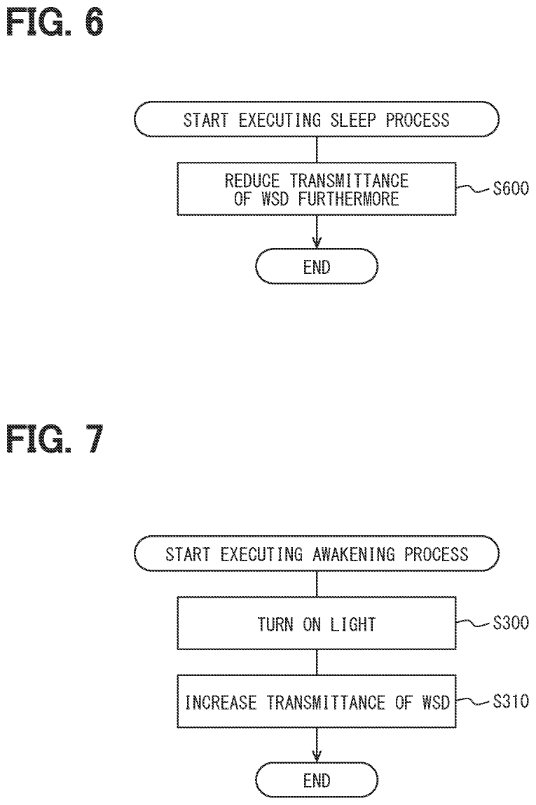

In S210, the sleep process shown in the flowchart of FIG. 6 is executed in the entire area of the WSD 17 (that is, the windshield 25). Specifically, in S600, the transmittance is lowered in the entire region of WSD17 as compared with the sleep onset mode. That is, in the sleep mode, the passenger compartment is darker than in the sleep onset mode so that the passenger can get a comfortable sleep.

On the other hand, in S220, the sleep process shown in the flowchart of FIG. 6 is executed in a part of the WSD 17 (that is, the windshield 25). Specifically, in S600, the transmittance of the region of the WSD 17 corresponding to the sleeping passenger is lowered as compared with the sleep onset mode. As a result, the external light that reaches the eyes of the sleeping passenger is further reduced as compared with the sleep mode, but the external light that reaches the eyes of other passengers is not reduced.

Subsequently, the processes of S110 to S140 and the process of S150 are executed in parallel. In S110, the facial expression, body temperature, and heartbeat of each passenger are acquired, and the sleep state of the passenger who has entered the sleep mode is confirmed based on the acquired facial expression, body temperature, and heartbeat of each passenger.

Subsequently, in S120, it is determined whether or not the sleeping passenger has taken a sufficient nap. For example, when a deep sleep is taken for a period of about 15 minutes, it is determined that a sufficient nap has been taken. When it is determined in S120 that a sufficient nap has not been taken, the process proceeds to S130, and when it is determined that a sufficient nap has been taken, the process proceeds to S140.

In S130, it is determined whether or not a nap can be safely taken to the destination. When it is determined in S130 that a nap can be taken, the process returns to S110, and when it is determined in S130 that the nap cannot be taken, the process proceeds to S140.

In S140, the normal awakening mode is turned on and the flowchart shown in FIG. 4 is executed. When a passenger sleeps for too long, the passenger may not be able to return to driving immediately after waking up. Therefore, when the passenger can take a sufficient nap, the passenger is awakened. First, in S200, it is determined whether or not the passengers to be awakened are a part of the passengers. In S200, when it is determined to awaken all the passengers, the process proceeds to S210, and when it is determined to awaken some passengers, the process proceeds to S220.

In S210, the awakening process shown in the flowchart of FIG. 7 is executed in the entire area of the WSD 17 (that is, the windshield 25). Specifically, in S300, the entire illuminator 18 is turned on to irradiate the faces of all the passengers with light.

Subsequently, in S310, the transmittance is increased to the standard transmittance in the entire region of the WSD 17. That is, the sleep mode is shifted to the normal mode. In this case, the transmittance may be gradually increased, or the transmittance may be increased to the standard transmittance at once. This increases the amount of outside light that reaches each passenger's eyes.

On the other hand, in S220, the awakening process shown in the flowchart of FIG. 7 is executed in a part of the WSD 17 (that is, the windshield 25). Specifically, in S300, the region of the irradiator 18 corresponding to the awakening passenger, that is, the light emitting member corresponding to the awakening passenger is turned on, and the light is irradiated toward the face of the awakening passenger. No light is applied to the faces of passengers who are not awakened.

Subsequently, in S310, the transmittance of the region of the WSD 17 corresponding to the awakening passenger is increased to the standard transmittance. The transmittance of the region of WSD17 corresponding to the unawakened passenger is not changed.

When the passenger other than the driver is awake and the driver is sleeping, a system may notify the passenger other than the driver that the driver needs to be awakened to wake up the driver by the passenger other than the driver. In this case, instead of irradiating the light with the illuminator 18, a passenger other than the driver is asked to wake up the driver, and the transmittance of the WSD 17 is increased. The notification to the passengers other than the driver may be notified by voice, or may be displayed and notified by the WSD 17.

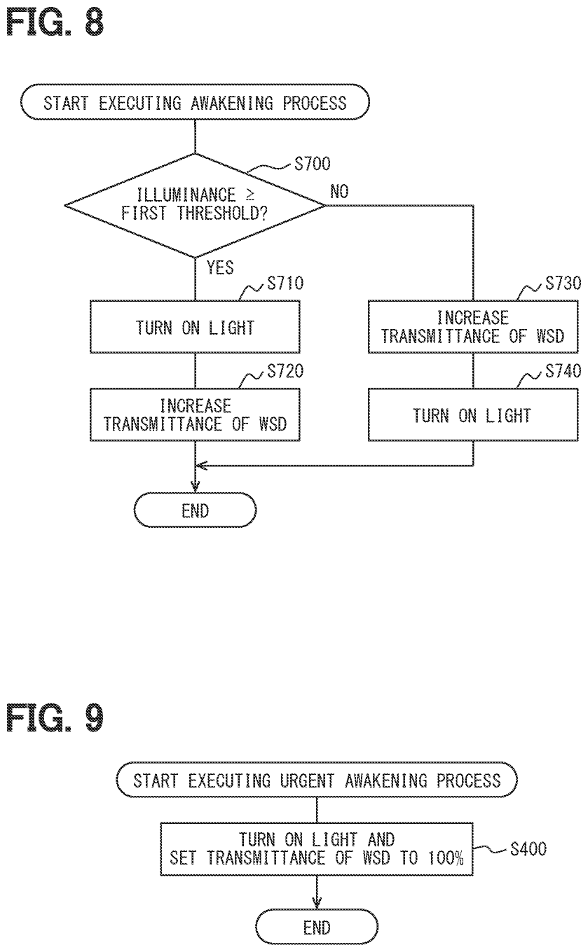

Here, in S210 and S220, the flowchart shown in FIG. 8 may be executed instead of the flowchart shown in FIG. 7.

First, in S700, the illuminance information is acquired and it is determined whether it is daytime or nighttime, that is, whether or not there is sunlight, according to whether or not the illuminance is larger than the first threshold value. In S700, when it is determined that the illuminance is equal to or higher than the first threshold value, the process proceeds to S710, and when it is determined that the illuminance is less than the first threshold value, the process proceeds to S730.

In S710, all or part of the illuminator 18 is turned on to irradiate all or part of the passenger's face with light.

Subsequently, in S720, the transmittance of all or part of the WSD 17 is increased to increase the light that reaches the eyes of all or part of the passengers. When the illuminance is equal to or higher than the first threshold value, the light of the illuminator 18 is weaker than the outside light, so the passenger can be comfortably awakened by controlling the light of the illuminator 18 to reach the passenger's eyes first.

On the other hand, in S730, the transmittance of all or part of the WSD 17 is increased to increase the light that reaches the eyes of all or part of the passengers.

Subsequently, in S740, all or part of the illuminator 18 is turned on to irradiate all or part of the passenger's face with light. When the illuminance is less than the first threshold value, the light of the illuminator 18 is stronger than the outside light, so the passenger can be comfortably awakened by controlling the outside light to reach the passenger's eyes first.

Returning to the flowchart of FIG. 3, in S150, it is determined whether or not there is a need for an emergency stop. Specifically, using the detection information from the peripheral monitoring sensor 11, it is determined whether or not the distance to the obstacle in front of the vehicle approaches the threshold value at which the emergency brake is activated. Alternatively, it is determined whether or not an emergency brake signal has been output. When it is determined in S150 that there is no need for an emergency stop, the process of S150 is repeatedly executed. When it is determined in S150 that an emergency stop is necessary, the process proceeds to S160.

In S160, the emergency awakening mode is turned on, and the emergency awakening process shown in the flowchart of FIG. 9 is executed. Specifically, in S400, the entire illuminator 18 (that is, all light emitting members) is turned on to irradiate light, and at the same time, the transmittance of the entire region of WSD 17 is increased to 100% at once. As a result, the inside of the vehicle compartment becomes bright at once. That is, in the event of an emergency of the autonomous driving vehicle 200, priority is given to promptly awakening the passenger rather than comfortably awakening the passenger. This is the end of this process.

(3. Effect)

According to the first embodiment described above, the following effects can be exhibited.

(1) The transmittance of WSD17 is reduced in order to promote the sleep of the passengers. By reducing the transmittance of the WSD 17, the incident of sunlight into the vehicle compartment is suppressed. Therefore, the passenger can fall asleep quickly and take a nap in the passenger compartment.

(2) The transmittance of WSD is increased in order to promote the awakening of the passengers. Increasing the transmittance of WSD increases the incidence of sunlight into the vehicle compartment. As a result, the passenger can be awakened.

(3) The passenger's biometric information is detected, and the detected biometric information is used to determine whether or not the passenger is in a state of drowsiness. Then, when it is determined that the passenger is in a state of drowsiness, the transmittance of WSD17 is lowered. Therefore, when the passenger is drowsy, it is possible to automatically construct a vehicle compartment environment in which it is easy for the passenger to fall asleep.

(4) Using biometric information, it is determined whether or not the passenger is in a state of awakening. When it is determined that the passenger is awakened, the transmittance of WSD17 is increased. Therefore, when the passenger is in a state where he/she should be awakened, it is possible to automatically construct a vehicle compartment environment in which the passenger is likely to be awakened. In addition, it is possible to suppress the occurrence of a situation in which the driver sleeps too much and cannot return to driving.

(5) When it is determined that the passenger is to be awakened, the transmittance of the WSD 17 is increased after the light of the illuminator 18 is irradiated. As a result, the burden on the passenger can be suppressed and the passenger can be awakened comfortably.

(6) When there is sunlight irradiation, the transmittance of WSD 17 is increased after irradiating the light of the illuminator 18 which is weaker than the outside light. On the other hand, when there is no irradiation of sunlight, the light of the illuminator 18 stronger than the outside light is irradiated after the transmittance of the WSD 17 is increased. As a result, the burden on the passenger can be suppressed according to the external environment, and the passenger can be comfortably awakened.

(7) In an emergency, the transmittance of WSD 17 can be increased to 100% at once at the same time as the light of the illuminator 18 irradiates. As a result, the interior of the vehicle compartment becomes bright at once, so that the passengers can be awakened quickly.

(8) By controlling the transmittance of the region of WSD17 corresponding to each passenger, it is possible to promote sleep or awakening for each passenger.

As a result, it does not cause discomfort to passengers who do not need to promote sleep or awakening.

(9) By irradiating the light of the illuminator 18 for each passenger, there is no discomfort to the occupant who does not need to be awakened.

(10) By changing the transmittance with time for each of the plurality of regions set in WSD 17, it is possible to encourage the passenger to sleep. In particular, when a heavy load is scheduled after the passenger gets off the vehicle, the passenger can be encouraged to sleep and the passenger can effectively take a nap.

(11) By lowering the transmittance of WSD17 to the minimum value, switching the WSD 17 to be a screen, and displaying an image that induces drowsiness, it is possible to encourage the passenger to sleep.

### Second Embodiment

### 1. Difference from First Embodiment

Since basic configuration of a second embodiment is the same as that of the first embodiment, the description of the common configuration will not be made, and the description will be made on the differences. The same reference numerals as in the first embodiment denote the same components, and reference is made to the preceding description.

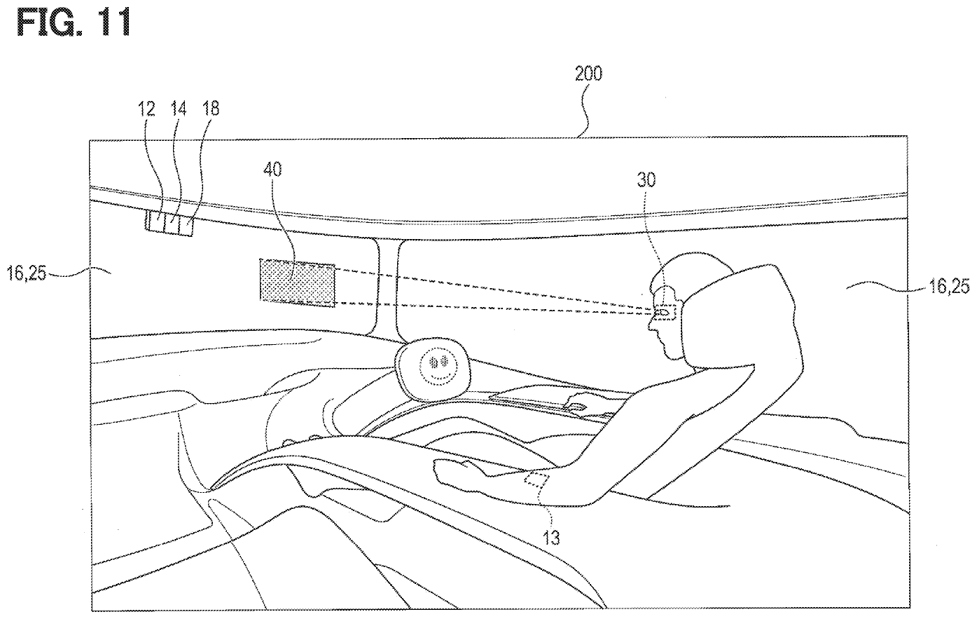

In the first embodiment described above, in the awakening process, the transmittance of the region of the WSD 17 corresponding to the passenger to be awakened is increased to the standard transmittance. On the other hand, the second embodiment differs from the first embodiment in that, as shown in FIG. 11, in the awakening process, the transmittance of the eye-corresponding region 40 of the WSD 17 is fixed at a value at which the illuminance required for awakening can be obtained, and the regions of the WSD 17 other than the eye-corresponding region 40 of the WSD 17 is increased to the standard transmittance. The eye-corresponding region 40 corresponds to the awakening passenger's eye region 30 in the WSD 17 (i.e., the windshield 25).

In this embodiment, the biometric information includes the passenger's eye area 30. The ECU 10 acquires the eye region 30 from the face image taken by the DSM 14. In the present embodiment, the DSM 14 corresponds to the observation device, and the face image corresponds to the observation information.

### 2. Awakening Process

Next, in the present embodiment, the awakening process executed by the ECU 10 will be described. In the present embodiment, the ECU 10 executes the same processing as in the first embodiment except for the processing of S140 in the vehicle compartment environment control process.

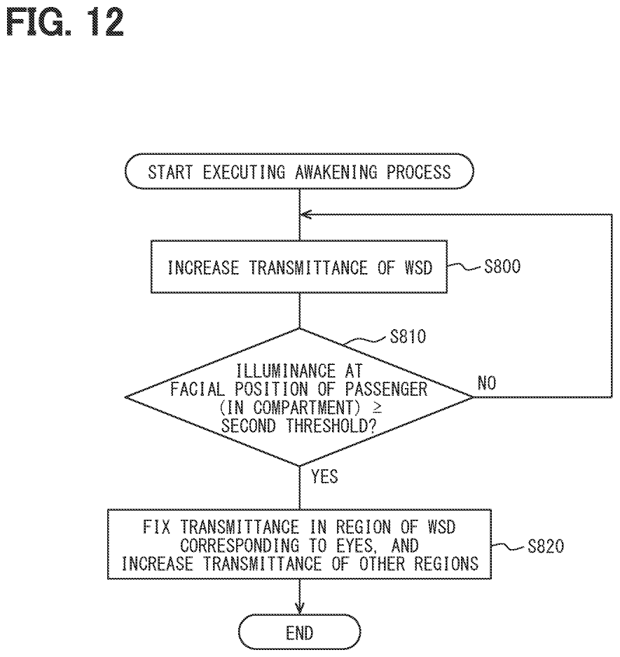

In this embodiment, the ECU 10 executes the flowchart shown in FIG. 4 when the normal awakening mode is turned on in S140. Then, in S210 and S220, the awakening process shown in FIG. 12 is executed.

First, in S800, the transmittance of WSD17 is gradually increased.

Subsequently, in S810, the illuminance information is acquired, and it is determined whether or not the illuminance at the position of the passengers face to be awakened, that is, the illuminance in the vehicle compartment is equal to or higher than the second threshold value. The second threshold is larger than the first threshold and smaller than the reference value. The second threshold is the illuminance required for the awakening of the passenger, for example, 2500 lx. When it is determined in S810 that the illuminance in the vehicle compartment is equal to or higher than the second threshold value, the process proceeds to S820, and when it is determined that the illuminance in the vehicle compartment is less than the second threshold value, the process returns to S800.

In S820, the eye region 30 is acquired from the facial image of the passenger to be awakened, the transmittance of the eye-corresponding region 40 of the WSD 17 (that is, the windshield 25) corresponding to the eye region 30 is fixed, and the transmittance is not increased from that value. That is, when the transmittance of the eye-corresponding region 40 of the WSD 17 reaches the second threshold value, the transmittance is fixed. The eye-corresponding region 40 is the incident range of the external light in the WSD 17, and corresponds to the incident range of the external light reaching the passenger's eye region 30. Further, in S820, as shown in FIG. 11, the transmittance of the region other than the eye-corresponding region 40 in WSD17 is increased to the reference value.

In this way, by suppressing the illuminance of the external light that reaches the passenger's eye region 30 to the minimum level at which the illuminance required for awakening can be obtained, it is possible to prevent the passenger's eye region 30 from suddenly brightening. As a result, it is possible to prevent the passenger from being dazzled by the sudden change in brightness. When it is necessary to switch the driving to the passenger after the passenger is awakened, the transmittance of the eye-corresponding region 40 of the WSD 17 may be gradually increased from a fixed value to a reference value. Further, when it is not necessary to switch the driving to the passenger after the passenger is awakened, the transmittance of the eye-corresponding region 40 of the WSD 17 may be maintained at a fixed value.

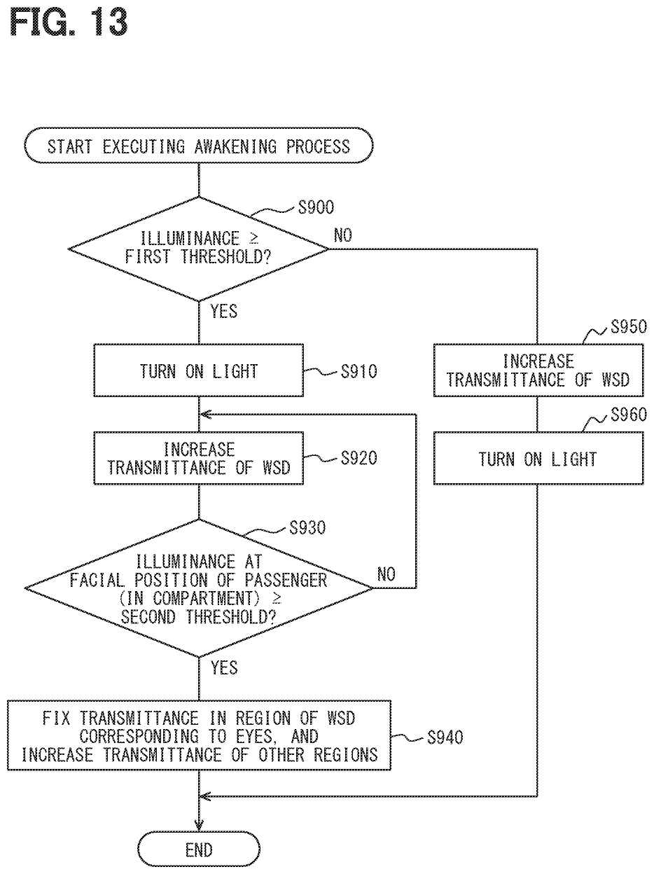

Here, in S210 and S220, the flowchart shown in FIG. 13 may be executed instead of the flowchart shown in FIG. 12.

First, in S900 to S920, the same processing as in S700 to S720 of the flowchart shown in FIG. 8 is executed.

Subsequently, in S930, the illuminance information is acquired, and it is determined whether or not the illuminance at the position of the passengers face to be awakened, that is, the illuminance in the vehicle compartment is equal to or higher than the second threshold value. The illuminance in the vehicle compartment here corresponds to the combined illuminance of both the external light incident from the WSD 17 and the irradiation light emitted from the illuminator 18.

When it is determined in S930 that the illuminance in the vehicle compartment is equal to or higher than the second threshold value, the process proceeds to S940, and when it is determined that the illuminance in the vehicle compartment is less than the second threshold value, the process returns to S920.

In S940, the same processing as in S820 of the flowchart shown in FIG. 12 is executed. As a result, the total illuminance of the outside light incident on the vehicle compartment from the WSD 17 and the irradiation light of the illuminator 18 is suppressed to the minimum necessary for awakening the passenger.

Further, in S950 and S960, the same processing as in S730 and S740 in the flowchart shown in FIG. 8 is executed. That is, in the flowchart shown in FIG. 13, a combination of the flowchart shown in FIG. 8 and the flowchart shown in FIG. 12 is executed.

According to the second embodiment described above, the following effects are provided in addition to the effects (1) to (4) and (6) to (11) of the first embodiment described above.

(12) When the passenger is awake, the illuminance of the light reaching the passenger's eye area 30 is suppressed to the minimum necessary for the passenger's awakening, so that it is suppressed for the passenger to feel a sudden change in brightness and to be dazzled.

### Other Embodiments

Although embodiments of the present disclosure have been described above, the present disclosure is not limited to the above-described embodiments but various modifications can be made.

(A) In the above embodiment, the illuminance amount information detected by the illuminance sensor 15 is used to determine whether it is daytime or nighttime, alternatively, the present disclosure may not be limited to this. For example, the ECU 10 may determine from the date and the current time whether it is daytime or nighttime, that is, whether or not there is sunlight. Alternatively, the ECU 10 may acquire the illuminance amount information by wireless communication with the information center or the roadside unit.

(B) The vehicle display control device and the technique according to the present disclosure may be achieved by a dedicated computer provided by constituting a processor and a memory programmed to execute one or more functions embodied by a computer program. Alternatively, the vehicle display control device and the technique according to the present disclosure may be achieved by a dedicated computer provided by constituting a processor with one or more dedicated hardware logic circuits. Alternatively, the vehicle display control device and the technique according to the present disclosure may be achieved using one or more dedicated computers constituted by a combination of the processor and the memory programmed to execute one or more functions and the processor with one or more hardware logic circuits. The computer program may also be stored on a computer readable non-transitory tangible recording medium as computer executable instructions. The technique for realizing the functions of the respective units included in the vehicle display control device does not necessarily need to include software, and all of the functions may be realized with the use of one or multiple hardware.

(C) The multiple functions of one component in the above embodiments may be realized by multiple components, or a function of one component may be realized by multiple components. Further, multiple functions of multiple elements may be implemented by one element, or one function implemented by multiple elements may be implemented by one element. In addition, a part of the configuration of the above embodiment may be omitted. Further, at least part of the configuration of the above-described embodiment may be added to or replaced with the configuration of another embodiment described above.

(D) In addition to the vehicle display control device described above, the present disclosure may be realized by various features such as a system having the vehicle display control device as a component, a program for operating a computer as the vehicle display control device, a non-transitory tangible storage medium such as a semiconductor memory storing this program, a display control method for a vehicle and the like.

The controllers and methods described in the present disclosure may be implemented by a special purpose computer created by configuring a memory and a processor programmed to execute one or more particular functions embodied in computer programs. Alternatively, the controllers and methods described in the present disclosure may be implemented by a special purpose computer created by configuring a processor provided by one or more special purpose hardware logic circuits. Alternatively, the controllers and methods described in the present disclosure may be implemented by one or more special purpose computers created by configuring a combination of a memory and a processor programmed to execute one or more particular functions and a processor provided by one or more hardware logic circuits. The computer programs may be stored, as instructions being executed by a computer, in a tangible non-transitory computer-readable medium.

It is noted that a flowchart or the processing of the flowchart in the present application includes sections (also referred to as steps), each of which is represented, for instance, as S10. Further, each section can be divided into several sub-sections while several sections can be combined into a single section. Furthermore, each of thus configured sections can be also referred to as a device, module, or means.

While the present disclosure has been described with reference to embodiments thereof, it is to be understood that the disclosure is not limited to the embodiments and constructions. The present disclosure is intended to cover various modification and equivalent arrangements. In addition, while the various combinations and configurations, other combinations and configurations, including more, less or only a single element, are also within the spirit and scope of the present disclosure.

What is claimed is:

1. A vehicular display control device mounted on an autonomous driving vehicle provided with a windshield display,
the windshield display being configured to change a transmittance of external light, the vehicular display control device comprising:
a transmittance control unit configured to reduce the transmittance in order to promote a sleep of at least one passenger.

2. The vehicular display control device according to claim 1, wherein:
the transmittance control unit is further configured to increase the transmittance in order to promote an awakening of the at least one passenger.

3. The vehicular display control device according to claim 1, wherein:
the autonomous driving vehicle includes a biosensor configured to detect biometric information of the at least one passenger,
the vehicular display control device further comprising:
a biometric information acquisition unit configured to acquire detection information detected by the biosensor; and
a drowsiness determination unit configured to determine whether the at least one passenger is in a state of drowsiness, using the detection information acquired by the biometric information acquisition unit, wherein:
the transmittance control unit is configured to reduce the transmittance when the drowsiness determination unit determines that the at least one passenger is in the state of drowsiness.

4. The vehicular display control device according to claim 1, wherein:
the autonomous driving vehicle includes a biosensor configured to detect biometric information of the at least one passenger,
the vehicular display control device further comprising:
a biometric information acquisition unit configured to acquire detection information detected by the biosensor; and
an awakening determination unit configured to determine whether the at least one passenger is in a state of awakening, using the detection information acquired by the biometric information acquisition unit, wherein:
the transmittance control unit is configured to increase the transmittance when the awakening determination unit determines that the at least one passenger is in the state of awakening.

5. The vehicular display control device according to claim 4, wherein:
the autonomous driving vehicle includes an observation device configured to observe the at least one passenger; and
the autonomous driving vehicle includes an illuminator installed in a passenger compartment and configured to detect illuminance,
the vehicular display control device further comprising:
an eye detection unit configured to detect an eye area of the at least one passenger based on observation information by the observation device, wherein:
when increasing the transmittance, the transmittance control unit fixes the transmittance in an eye-corresponding region in response to a feature that the illuminance detected by the illuminator reaches a predetermined value; and
the eye-corresponding region in the windshield display corresponds the eye area detected by the eye detection unit.

6. The vehicular display control device according to claim 5, wherein:
the transmittance control unit is configured to increase the transmittance in a region of the windshield display other than the eye-corresponding region from a fixed value; and
the fixed value is a value of the transmittance of the eye-corresponding region which is fixed in response that the illuminance reaches the predetermined value.

7. The vehicular display control device according to claim 4, wherein:
the autonomous driving vehicle includes an illuminator,
the vehicular display control device further comprising:
a lighting control unit configured to irradiate a light of the illuminator toward the at least one passenger when the awakening determination unit determines that the at least one passenger is in the state of awakening, wherein:
the transmittance control unit is configured to increase the transmittance after the lighting control unit irradiates the light of the illuminator.

8. The vehicular display control device according to claim 4, wherein:
the autonomous driving vehicle includes an illuminator,
the vehicular display control device further comprising:
an external information acquisition unit configured to acquire environmental information outside the vehicle; and
a lighting control unit configured to irradiate a light of the illuminator toward the at least one passenger when the awakening determination unit determines that the at least one passenger is in the state of awakening, wherein:
the transmittance control unit is configured to increase the transmittance according to the environmental information acquired by the external information acquisition unit either after or before an irradiation of a light of the illuminator controlled by the lighting control unit.

9. The vehicular display control device according to claim 7, wherein:
the transmittance control unit increases the transmittance while simultaneously irradiating the light of the illuminator controlled by the lighting control unit in response to an emergency of the autonomous driving vehicle.

10. The vehicular display control device according to claim 1, wherein:
the at least one passenger includes a plurality of passengers;
the transmittance control unit is configured to control the transmittance of a passenger-corresponding display region corresponding to each of the plurality of passengers according to respective states of the plurality of passengers; and
a passenger-corresponding display area is an area of the windshield display corresponding to each of the plurality of passengers.

11. The vehicular display control device according to claim 7, wherein:
the at least one passenger includes a plurality of passengers;
the lighting control unit irradiates light from a passenger-corresponding illuminator area corresponding to each of the plurality of passengers toward each of the plurality of passengers according to respective states of the plurality of passengers; and
the passenger-corresponding illuminator area is an area of the illuminator corresponding to each of the plurality of passengers.

12. The vehicular display control device according to claim 1, wherein:
the transmittance control unit sets a plurality of regions on the windshield display in order to promote a sleep of the at least one passenger; and
the transmittance control unit changes the transmittance for each of the plurality of regions with time.

13. The vehicular display control device according to claim 1, further comprising:
an image display unit configured to display an image that induces drowsiness on the windshield display in order to promote the sleep of the at least one passenger.
